(19) United States
(12) Patent Application Publication
(10) Pub. No.: US 2011/0305757 A1
(43) Pub. Date: Dec. 15, 2011

(54) NEW PHARMACEUTICAL COMBINATIONS

(75) Inventors: Umit Cifter, Istanbul (TR); Ali Turkyilmaz, Istanbul (TR); Onur Mutlu, Istanbul (TR); Gaye Ramazanoglu, Istanbul (TR)

(73) Assignee: Sanovel Ilac Sanayi Ve Ticaret Anonim Sirketi, Istanbul (TR)

(21) Appl. No.: 13/159,049

(22) Filed: Jun. 13, 2011

(30) Foreign Application Priority Data
Jun. 11, 2010 (TR) 201004754

(51) Int. Cl. A61K 9/28; A61K 31/549; B29C 67/24; A61P 9/12; B29C 67/00; A61K 31/4245; A61K 9/24

(52) U.S. Cl. 424/474; 514/364; 514/223.5; 424/464; 264/117; 264/123

(57) ABSTRACT

A pharmaceutical composition including a compound of Formula I (Compound I) or pharmaceutically acceptable salts thereof and one or more diuretics as effective components, wherein said one or more diuretics are selected from thiazide derivatives. Methods for preparing the pharmaceutical compound including Compound I and thiazide derivatives and its use for preventing or treating hypertension in mammals, particularly in humans.

### CROSS REFERENCE TO RELATED APPLICATION

This application is based upon Turkish Patent Application No. TR201004754, filed Jun. 11, 2010, under relevant sections of 35 USC §119, the entire contents of that application being incorporated by reference herein.

### TECHNICAL FIELD

A novel pharmaceutical composition comprising a compound of 2-ethoxy-1-((2′-(5-oxo-2,5-dihydro-1,2,4-oxadiazol-3yl)-biphenyl-4-yl)methyl)-1H-benzimidazole-7-carboxylic acid (Compound I) or pharmaceutically acceptable salts thereof and one or more diuretics as effective components, wherein said one or more diuretics are selected from thiazide derivatives.

Furthermore, methods are provided for preparing the pharmaceutical composition comprising Compound I and thiazide derivatives and its use for preventing or treating hypertension in mammals, particularly in humans.

### BACKGROUND

Hypertension affects about 20% of the adult population in developed countries. In the adult population aged 60 years or older, this percentage increases to about 60% to 70% in general. Hypertension is also associated with an increased risk of other physiological complications including stroke, myocardial infarction, atrial fibrillation, heart failure, peripheral vascular disease and renal impairment. Although a number of anti-hypertensive drugs are available in various pharmacological categories, the efficacy and safety of such drugs can vary from patient to patient and in this regard new treatments are still a desired subject.

Azilsartan, which has a chemical name as 2-ethoxy-1-((2′-(5-oxo-2,5-dihydro-1,2,4-oxadiazol-3yl)-biphenyl-4-yl)methyl)-1H-benzimidazole-7-carboxylic acid (hereinafter referred as “Compound I”) is a novel angiotensin II receptor antagonist having a chemical structure as shown in the following Formula I:

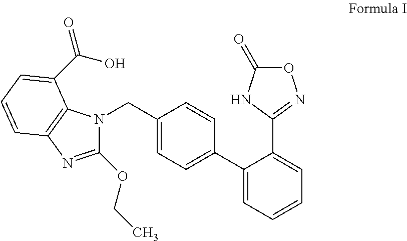

Angiotensin II receptor antagonists are used in the management of hypertension. These antagonists they may have a particular role in patients who develop cough with ACE inhibitors. Some are also used in diabetic nephropathy and in the management of heart failure. They act mainly by selective blockade of AT1 receptors, thus reducing the pressor effects of angiotension II. Known angiotension receptor II antagonists from the prior art include candesartan, eprosartan, irbesartan, losartan, olmesartan, telmisartan and valsartan.

Diuretics are orally administered in the treatment of edema and hypertension. Well known diuretics are thiazides which are known moderately potent diuretics and exert their diuretic effect by reducing the reabsorbtion of electrolytes from the renal tubules, thereby increasing the excretion of sodium and chloride ions, and consequently of water.

Hydrochlorothiazide is a thiazide diuretic and its chemical name is 2H-1,2,4-Benzothiadiazine-7-sulfonamide, 6-chloro-3,4-dihydro-,1,1-dioxide having the following structure (Formula II).

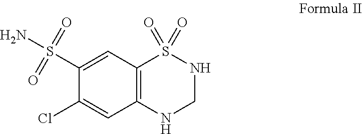

Thiazide diuretics are used in the treatment of hypertension, either alone or in combination with other antihypertensives. They are also used to treat edema associated with heart failure and with renal and hepatic disorders. Thiazide diuretics, particularly hydrochlorothiazide, may enhance the effect of other antihypertensives, particularly first-dose hypotension that occurs with angiotensin II receptor antagonists.

It is known that co-administration of an angiotensin II receptor antagonist and a diuretic is an effective therapy for the prevention or treatment of hypertension.

However, the effects of a pharmaceutical composition comprising a specific angiotensin II receptor antagonist, such as Compound I and a diuretic selected from thiazide derivatives such as hydrochlorothiazide remain unknown.

The main challenges when combining two or more molecules in the same pharmaceutical form are: (a) to guarantee the chemico-physical compatibility between the different active ingredients and/or between the active ingredients and the excipients used; (b) to insure the therapeutical compatibility between the two active ingredients regarding their pharmacokinetic and/or pharmaceutical properties in order that the posology of the combined composition allows to obtain safe and efficient plasma levels of both pharmacological agents; and (c) to insure lower incidence of side effects.

Considering this, combinations of various drugs have been investigated and it has been found that a pharmaceutical composition containing a specific angiotensin II receptor antagonist, such as Compound I, and one or more diuretics selected from thiazide derivatives such as hydrochlorothiazide exerts excellent anti-hypertensive effects and hence is useful as a preventative and/or therapeutic agent for hypertension.

Furthermore, it is known that obtaining an adequate content uniformity is an important issue, especially in pharmaceutical compositions comprising more than one active ingredient. Because it is difficult to homogenize the active ingredients, particularly if they have low doses in final dosage form and some other difficulties that may occur when compressing them during manufacturing process, this may cause inadequate content uniformity of the final dosage forms. As a result of this, severe problems may occur during therapy using these drugs.

It is also known in the pharmaceutical field that, when formulating a composition comprising more than one pharmaceutical active ingredient for administration to those in need of therapy, there is a challenge to ensure an even distribution of the pharmaceutically active ingredients throughout the pharmaceutical excipients to ensure a proper dosage and homogeneity. Therefore, it would be desirable to provide improved processes for preparing solid oral dosage forms that have an adequate content uniformity, that disperse well upon oral administration and are robust enough to remain stable during the shelf-life.

Therefore, there is need in the art for pharmaceutical compositions of Compound I or pharmaceutically acceptable salts thereof and a thiazide diuretic, particularly hydrochlorothiazide for oral administration, which has an adequate content uniformity causing a good dispersion upon oral administration and high bioavailability with improved manufacturing processes and stability and a robust final dosage form for their preparation and use thereof.

### SUMMARY AND DETAILED DESCRIPTION

It is desired to provide new pharmaceutical compositions comprising a specific angiotensin II receptor antagonist such as Azilsartan which has a chemical name as 2-ethoxy-1-((2′-(5-oxo-2,5-dihydro-1,2,4-oxadiazol-3yl)-biphenyl-4-yl)methyl)-1H-benzimidazole-7-carboxylic acid (hereinafter referred as “Compound I”) and one or more diuretics, such as thiazide derivatives, as effective components which overcomes the above described problems in prior art and have additive advantages over them.

According to one aspect, the pharmaceutical compositions of this formulation are suitable for the prevention or treating hypertension in mammals, particularly in humans.

It is also desired to obtain adequate content uniformity of dosage forms of Compound I or pharmaceutically acceptable salts thereof and one or more diuretics selected from thiazide derivatives such as hydrochlorothiazide with using adequate excipients.

It is also desired to provide pharmaceutical compositions of Compound I or pharmaceutically acceptable salts thereof with hydrochlorothiazide which is robust (e.g. adequate hardness, low friability) enough to be processed in high speed tablet pressing machines and shipped in low cost packages.

It is further desired to provide stable pharmaceutical compositions having high bioavailability throughout their shelf-life.

Yet another desire is to provide an improved process which is simple, cost-effective and time saving for preparing the pharmaceutical composition of Compound I or pharmaceutically acceptable salts thereof and one or more thiazide diuretics such as hydrochlorothiazide.

According to one aspect, said thiazide derivatives are selected from the group comprising hydrochlorothiazide, methylclothiazide, benzylhydrochlorothiazide, trichlormethiazide, cyclopenthiazide, polythiazide, ethiazide, cyclothiazide, bendroflumethiazide or hydroflumethiazide or mixtures thereof; preferably, the thiazide derivative is hydrochlorothiazide.

Investigations therefore were made to provide a fixed dose drug combination comprising an angiotensin II receptor antagonist, such as Compound I or pharmaceutically acceptable salts thereof, and one or more diuretics selected from thiazide derivatives such as hydrochlorothiazide. This combination drug should display an immediate drug release profile combined with adequate stability. A fixed dose combination of drugs intended for immediate release can be prepared by either making a powder mixture or a co-granulate of the two active ingredients with the necessary excipients, normally keeping the basic formulation of the corresponding mono-drug preparation and simply adding the second drug component.

Accordingly, the specific angiotensin II receptor antagonist such as Compound I and diuretics selected from thiazide derivatives such as hydrochlorothiazide exert better therapeutic efficacy by combined administration rather than when used separately. In addition, combinations of angiotensin II receptor antagonist and diuretics selected from thiazide derivatives such as hydrochlorothiazide are more suitable, in terms of safety or efficacy, than the administration of a single product.

However, particularly with a combination of Compound I or pharmaceutically acceptable salts thereof with hydrochlorothiazide, this approach may not feasible due to the difficulties such as Compound I is difficult to formulate and heretofore it has not been possible to make oral formulations in the form of tablets in homogenous and robust way.

We have surprisingly found that, the selection of excipients therefore has an importance to obtain an adequate content uniformity and robustness. Thus, at least one disintegrant with one or more pharmaceutically acceptable excipients can be used to obtain adequate content uniformity. The disintegrant is selected from the group comprising crospovidone, sodium starch glycolate, croscarmellose sodium, low-substituted hydroxypropyl cellulose, starch derivatives and the like or mixtures thereof; preferably, the selected disintegrant is crospovidone and/or sodium starch glycolate.

Thus, crospovidone has physical and chemical properties that make it ideal for constituting the appropriate disintegrant for this formulation. Because crospovidone particles have a very different appearance from those of the other disintegrants, crospovidone particles seem to consist of aggregates of smaller particles that are fused together. This aggregation gives crospovidone a spongy, highly porous appearance and it swells very little, yet takes water into its network quite rapidly. This helps crospovidone to dissolve easily and quickly in a little amount of water and makes its disintegrating rate much faster than other related excipients.

According to one aspect, crospovidone is present in an amount of between 0.1.0 to 30.0% by weight, preferably in an amount of 1.0 to 20.0% by weight of the total formulation.

In the prior art, pharmaceutical compositions of angiotensin II receptor antagonists with thiazide diuretics mostly contain corn starch and mannitol with other common known excipients. However unmodified starch, such as corn starch, has poor flow characteristics and tends to increase tablet friability and capping if used in high concentrations.

We have found that solid oral dosage forms such as tablets, when prepared with sodium starch glycolate possess good storage properties. Additionally, the improved flow and lubricity characteristics of sodium starch glycolate can impart further benefit to the formulation in a very cost-effective manner. The reason for the selection of sodium starch glycolate is mainly because of its high disintegrant properties besides its binding properties during granulation step in manufacturing process. Therefore both properties can be used advantageously in one step.

As noted said above, the selection of excipients is very important to obtain robust final dosage forms when it is in tablet form. According to this object, investigations have been made and it has been found that sodium starch glycolate is one of the best excipients to obtain an adequate robustness in the final tablet dosage forms of the pharmaceutical compositions described herein.

Surprisingly, a synergistic effect is observed over the distribution of the doses of Compound I and hydrochlorothiazide throughout other pharmaceutical excipients when crospovidone and sodium starch glycolate is used in a specific weight ratio wherein the weight ratio of crospovidone to sodium starch glycolate is between 30:1 to 1:100 by weight; preferably, it is 20:1 to 1:10 by weight and more preferably 10:1 to 1:1 by weight of total composition. Thus, a proper dosage and homogeneity is ensured and the final dosage forms have high and adequate content uniformity. According to this embodiment, the final dosage form of the pharmaceutical composition of Compound I and hydrochlorothiazide has a content uniformity of less than 2.0% RSD (Relative Standard Deviation). Preferably the RSD is less than 1.0% RSD.

According to another embodiment, the pharmaceutical composition further comprise one or more pharmaceutical acceptable excipients which are selected from the group comprising fillers & diluents, binders, lubricants, glidants, coloring agents, coating agents or mixtures thereof.

In one embodiment, suitable fillers & diluents are selected from the group comprising microcrystalline cellulose, lactose, sucrose, glucose, sorbitol, inorganic salts, dibasic calcium phosphate dihydrate and the like or mixtures thereof; preferably, the filler & diluents is microcrystalline cellulose.

In addition, it has found that magnesium stearate has some disadvantages despite being a good lubricant and because of this it is used in small quantities during the drug manufacturing process. Magnesium stearate is practically insoluble in water and because of this hydrophobic characteristic it may retard the dissolution of a drug from a solid dosage form such as tablet or capsule. Tablet and especially capsule dissolution is sensitive to both the amount of magnesium stearate in the formulation and the blending time. Blending time should be limited. Long blending times can result in the formulation of hydrophobic powder beds that do not disperse easily and overblending can cause compaction problems. Tablet dissolution rate and crushing strength decreased as the time of blending increased; and magnesium stearate may also increase tablet friability. Blending times with magnesium stearate should therefore be carefully controlled.

Thus, sodium stearyl fumarate is found to be an extremely effective lubricant and less hydrophobic than magnesium stearate, having a less retardant effect on tablet dissolution than magnesium stearate. Sodium stearyl fumarate also does not have the over blending problems seen with magnesium stearate.

According to the above embodiments, the pharmaceutical composition of the present formulation is free of magnesium stearate.

As it is mentioned above, a primary desire is to develop pharmaceutical compositions having optimal mechanical strength. The present formulation addresses this need and discloses formulations which have a good mechanical strength. These tablets are robust (e.g., low friability, adequate hardness) enough to be processed in high speed tablet pressing machines and shipped in low cost packages, and at the same time retain good dissolution properties. These pharmaceutical compositions have an adequate bioavailability and stable throughout their shelf-life.

Surprisingly, it has found that when microcrystalline cellulose and sodium stearyl fumarate are used together with Compound I or pharmaceutically acceptable salts thereof with hydrochlorothiazide, the pharmaceutical compositions of this formulation has better storage stability and results a synergistic effect over mechanical strength, such as having better compressibility with less friability. Thus, robust tablet formulations are obtained in the final dosage forms, when the weight ratio of microcrystalline cellulose to sodium stearyl fumarate is between 100:1 and 1:100 by weight; preferably, when it is between 50:1 and 1:10 by weight and even more preferably it is 35:1 and 1:1 by weight of the total formulation; said amount making it possible to significantly improve compressibility and reduce friability. Higher quantities may have negative mechanical strength of the formulation and lower quantities may worsen the stability.

According to this embodiment, the final tablet dosage forms have a hardness of between 5 to 300 Newton; preferably between 20 to 150 Newton and the friability of the final tablet dosage forms is less than 1%. The disintegration time of the final dosage forms are less than 2 minutes.

Furthermore, the pharmaceutical compositions obtained are stable both to the manufacturing process and during storage, e.g., a long-term shelf-life of 24 months or more at ambient temperature and in its original packaging, e.g. sealed aluminium blister packs.

In another embodiment, suitable binders are selected from the group comprising starches such as pregelatinized or modified cellulose derivatives such as hydroxypropyl methyl cellulose, hydroxypropyl cellulose, carboxy methyl cellulose, methyl cellulose; polyvinylpyrrolidone and the like or mixtures thereof.

According to the embodiments described above, suitable lubricants are selected from the group comprising at least one of sodium stearyl fumarate, polyethylene glycol, stearic acid, metal stearates, talc, waxes, boric acid, hydrogenated vegetable oils, sodium chloride benzoate and acetate, sodium or magnesium lauryl sulfate and the like or mixtures thereof; preferably the lubricant is sodium stearyl fumarate.

According to one embodiment, suitable glidants are selected from the group comprising colloidal silicon dioxide; silicates such as aluminium, calcium and magnesium, talc and the like or mixtures thereof; preferably the glidant is colloidal silicon dioxide.

According to one embodiment, suitable coloring agents are selected from the group comprising ferric oxide (red, yellow, black or mixtures) and Food & Drug Cosmetic Dyes and the like or mixtures thereof.

According to one embodiment, suitable coating agents are selected from the group comprising hydroxypropyl cellulose, hydroxypropyl methyl cellulose, methyl cellulose, polyethylene glycol, titanium dioxide, polyvinyl acetate phthalate, hydroxyproplyl methylcellulose phthalate, methacrylic acid copolymers and the like or mixtures thereof.

In one aspect, the pharmaceutical composition of Compound I is present in an amount of between 0.1 to 60.0% by weight; preferably it is present in an amount of between 0.1 to 50.0% by weight of the total composition, and hydrochlorothiazide is present in an amount of 0.1 to 40.0% by weight of total composition.

In a preferred embodiment, the pharmaceutical composition of Compound I or a pharmaceutically acceptable salt is in the form of medoxomil salt.

In the present composition, the specific angiotensin II receptor antagonist, such as Compound I and diuretics selected from thiazides such as hydrochlorothiazide are simultaneously administered, or separately or sequentially administered as described above.

In particular, the dosage form of the present composition is a solid dosage form such as tablets, capsules, powders, sachets, etc. The preferred dosage form is in tablet or capsule form.

Yet a further stated desire is to provide a pharmaceutical composition in the form of a bilayer tablet comprising Compound I or pharmaceutically acceptable salts in one layer and hydrochlorothiazide in second layer.

Furthermore, the bilayer tablet dosage form may comprise a coating.

The pharmaceutical compositions described herein may be prepared by conventional technology well known to those skilled in the art such as wet granulation, dry granulation and direct compression and the like.

One preferred wet granulation process for preparing the pharmaceutical composition of Compound I or pharmaceutical acceptable salts thereof and hydrochlorothiazide comprises the following steps:
a) dissolving Compound I and hydrochlorothiazide with half part of sodium starch glycolate and half part of crospovidone in organic solvent to form a solution;
b) while the solution is mixing, microcrystalline cellulose is added and blended in a high-shear granulator to form granules;
c) sieving and drying the wet granules and milling the dried granules;
d) adding colloidal silicon dioxide and the remaining part of sodium starch glycolate and crospovidone and mixing them;
e) adding sodium stearyl fumarate to this mixture and blending them until obtaining a homogenous powder mixture; and
f) compressing the blended mixture to form tablets or filling the powder mixture into capsules.

Another preferred wet granulation process for preparing the pharmaceutical composition of Compound I or pharmaceutical acceptable salts thereof and hydrochlorothiazide comprises the following steps:
a) dissolving Compound I and hydrochlorothiazide with microcrystalline cellulose, half part of sodium starch glycolate and half part of crospovidone in organic solvent to form a solution in a fluid-bed granulator;
b) sieving and drying the wet granules and milling the dried granules;
c) adding colloidal silicon dioxide and the rest of sodium starch glycolate and crospovidone and mixing them;
d) adding sodium stearyl fumarate to this mixture and blending them until obtaining a homogenous powder mixture; and
e) compressing the blended mixture to form tablets or filling the powder mixture into capsules.

A preferred dry granulation process for preparing the pharmaceutical composition of Compound I or pharmaceutical acceptable salts thereof and hydrochlorothiazide comprises the following steps:
a) mixing Compound I and hydrochlorothiazide with half part of microcrystalline cellulose, half part of sodium starch glycolate, half part of crospovidone and half part of colloidal silicon dioxide;
b) pressing them with the help of a compactor;
c) sieving these powder mixture;
d) adding the rest of microcrystalline cellulose, sodium starch glycolate, crospovidone and colloidal silicon dioxide and mixing them;
e) adding sodium stearyl fumarate to this mixture and blending them until obtaining a homogenous powder mixture; and
f) compressing the blended mixture to form tablets or filling the powder mixture into capsules.

A preferred direct compression process for preparing the pharmaceutical composition of Compound I or pharmaceutical acceptable salts thereof and hydrochlorothiazide comprises the following steps:
a) mixing Compound I and hydrochlorothiazide with microcrystalline cellulose, sodium starch glycolate, crospovidone and colloidal silicon dioxide for 15 to 30 min;
b) adding sodium stearyl fumarate to this mixture and blending them until obtaining a homogenous powder mixture; and
c) compressing the final powder mixture to form tablets or filling the powder mixture into capsules.

The pharmaceutical compositions described herein are suitable for preventing or treating hypertension in mammals, particularly in humans.

According to one main aspect, an adequate content uniformity with robust final dosage forms is obtained, for which this oral pharmaceutical composition has been designed, the composition being made up of the following:

a) 0.1 to 60.0% of compound I or pharmaceutically acceptable salts thereof;

b) 0.1 to 40.0% of hydrochlorothiazide;

c) 1.0 to 90.0% of microcrystalline cellulose;

d) 0.1 to 30.0% of crospovidone;

e) 0.01 to 50.0% of sodium starch glycolate;

f) 0.01 to 20.0. % of sodium stearyl fumarate; and

g) 0.01 to 15.0% of colloidal silicon dioxide.

Reference is made to the following example. Although the example is not intended to limit the scope of the present formulation, it should be considered in the light of the description detailed above. It will be apparent to those skilled in the art that many modifications, both to materials and methods, may be practiced without departing from the scope of the composition.

### EXAMPLE 1

a) 4.0% of compound I or pharmaceutically acceptable salts thereof

b) 5.0% of hydrochlorothiazide

c) 64.5% of microcrystalline cellulose

d) 20.0% of crospovidone

e) 4.0% of sodium starch glycolate

f) 2.0% of sodium stearyl fumarate

g) 0.5% of colloidal silicon dioxide

The formulation of this example is manufactured according to the process described above in the description.

### EXAMPLE 2

a) 4.0% of compound I or pharmaceutically acceptable salts thereof

b) 10.0% of hydrochlorothiazide

c) 64.0% of microcrystalline cellulose

d) 15% of crospovidone

e) 4% of sodium starch glycolate

f) 2.0% of sodium stearyl fumarate

g) 1.0% of colloidal silicon dioxide

The formulation of this example is manufactured according to the process described above in the description.

### EXAMPLE 3

a) 25.0% of compound I or pharmaceutically acceptable salts thereof

b) 12.50% of hydrochlorothiazide

c) 40.50% of microcrystalline cellulose

d) 15% of crospovidone

e) 4% of sodium starch glycolate

f) 2.0% of sodium stearyl fumarate

g) 1.0% of colloidal silicon dioxide

The formulation of this example is manufactured according to the process described above in the description.

### EXAMPLE 4

### Content Uniformity Test

The pharmaceutical composition of this present example (Example 1), was tested for the content uniformity against a reference product which is including mannitol, corn starch and magnesium stearate as some of the excipients instead of sodium starch glycolate, crospovidone, microcrystalline cellulose and sodium stearyl fumarate. The results are shown below in Table 1.

TABLE 1
Content Uniformity test results (%)

| | | | Reference | |
|---|---|---|---|---|
| | Example 1 | Example 1 | Product | Reference Product |
| Sample | Compound I | Hydrochlorothiazide | Compound I | Hydrochlorothiazide |
| No | content | content | content | content |
| 1 | 101.18 | 101.53 | 108.41 | 104.54 |
| 2 | 99.63 | 99.52 | 97.58 | 89.73 |
| 3 | 100.45 | 100.08 | 95.55 | 102.11 |
| 4 | 98.52 | 101.09 | 97.32 | 96.03 |
| 5 | 100.15 | 98.95 | 103.8 | 93.5 |
| 6 | 99.66 | 98.75 | 95.19 | 96.2 |
| 7 | 99.75 | 101.25 | 87.66 | 91.8 |
| 8 | 98.89 | 99.89 | 96.84 | 97.15 |
| 9 | 98.95 | 100.05 | 106.6 | 100.5 |
| 10 | 101.3 | 101.07 | 100.2 | 96.2 |
| mean | 99.848 | 100.218 | 98.915 | 96.776 |
| SD | 0.94 | 0.98 | 6.10 | 4.58 |
| RSD % | 0.94 | 0.98 | 6.17 | 4.74 |
| Min. | 98.52 | 98.75 | 87.66 | 89.73 |
| Max. | 101.3 | 101.53 | 108.41 | 104.54 |

Reference Product: Compound I, Hydrochlorothiazide as active ingredients and mannitol, corn starch, povidone, magnesium stearate and colloidal silicon dioxide as inactive ingredients.

### EXAMPLE 5

### Dissolution Profile Test

The pharmaceutical composition of this present example (Example 1), was tested by its dissolution profile in phosphate buffer at pH 7.8 and 37° C. using a USP apparatus 2 rotating at 50 RPM against the reference product which is mentioned in Example 4. The results are shown below in Table 2.

TABLE 2
Dissolution profile test results (%)

| | Example 1 | Example 1 | Reference Product | Reference Product |
|---|---|---|---|---|
| Time | Compound I | Hydrochlorothiazide | Compound I | Hydrochlorothiazide |
| (min.) | content (%) | content (%) | content (%) | content (%) |
| 5 | 75 | 80 | 51 | 60 |
| 10 | 85 | 88 | 72 | 78 |
| 15 | 90 | 92 | 80 | 83 |
| 20 | 95 | 95 | 85 | 88 |
| 30 | 97 | 98 | 92 | 94 |
| 45 | 99 | 100 | 95 | 96 |
| 60 | 101 | 101 | 98 | 99 |

### EXAMPLE 6

### Disintegration Test

The pharmaceutical composition of this present embodiment (Examples 1, 2 and 3), was tested by its disintegration against the reference product which is mentioned in Example 4. The results are shown below in Table 3.

TABLE 3
Disintegration test results (min.)

| | Example 1 | Example 2 | Example 3 | Reference product |
|---|---|---|---|---|
| Time (min.) | 1.50 | 1.60 | 1.75 | 3.50 |

1. A pharmaceutical composition comprising 2-ethoxy-1-((2′-(5-oxo-2,5-dihydro-1,2,4-oxadiazol-3yl)-biphenyl-4-yl)methyl)-1H-benzimidazole-7-carboxylic acid (Compound I) or pharmaceutically acceptable salts thereof and one or more diuretics as effective components, wherein said one or more diuretics are selected from thiazide derivatives.

2. The pharmaceutical composition according to claim 1, wherein the thiazide derivatives are selected from the group comprising at leat one of hydrochlorothiazide, methylclothiazide, benzylhydrochlorothiazide, trichlormethiazide, cyclopenthiazide, polythiazide, ethiazide, cyclothiazide, bendroflumethiazide and hydroflumethiazide and mixtures thereof.

3. The pharmaceutical composition of claim 1, wherein the thiazide derivative is hydrochlorothiazide, preferably the thiazide is hydrochlorothiazide.

4. The pharmaceutical composition according to claim 1, wherein the Compound I or pharmaceutically acceptable salts thereof is present in an amount of between 0.1 and 60.0% by weight and hydrochlorothiazide is present in an amount of 0.1 and 40.0% by weight.

5. The pharmaceutical composition according to claim 1, wherein the pharmaceutically acceptable salt of Compound I is medoxomil.

6. The pharmaceutical composition according to claim 1, further comprising a disintegrant and at least one excipient.

7. The pharmaceutical composition according to claim 6, wherein the disintegrant is selected from the group comprising at least one of crospovidone, sodium starch glycolate croscarmellose sodium, low-substituted hydroxypropyl cellulose and the like and mixtures thereof.

8. The pharmaceutical composition of claim 6, wherein the disintegrant is at least one of crospovidone and sodium starch glycolate.

9. The pharmaceutical composition according to claim 7, wherein crospovidone is present in an amount of between 0.10 to 30.0% by weight of total composition.

10. The pharmaceutical composition according to claim 8, wherein the weight ratio of crospovidone to sodium starch glycolate is between 30:1 and 1:100 by weight of total composition.

11. The pharmaceutical composition according to claim 6, wherein said at least one excipient is selected from the group comprising fillers & diluents, lubricants, glidants, binders, coloring agents, coating agents and mixtures thereof.

12. The pharmaceutical composition according to claim 11, wherein the filler & diluent is selected from the group comprising microcrystalline cellulose, lactose, sugars, inorganic salts, dibasic calcium phosphate dihydrate and the like and mixtures thereof.

13. The pharmaceutical composition according to claim 11, wherein the filler & diluents is microcrystalline cellulose.

14. The pharmaceutical composition according to claim 11, wherein the lubricant is selected from the group comprising at least one of sodium stearyl fumarate, polyethylene glycol, stearic acid, metal stearates, talc, boric acid, hydrogenated vegetable oils, sodium chloride benzoate and acetate, sodium or magnesium lauryl sulfate and the like and mixtures thereof.

15. The pharmaceutical composition according to claim 11, wherein the lubricant is preferably sodium stearyl fumarate.

16. The pharmaceutical composition according to claim 14, wherein the filler and lubricant is microcrystalline cellulose and the weight ratio of microcrystalline cellulose to sodium stearyl fumarate is between 100:1 and 1:100 by weight of the total formulation.

17. The pharmaceutical composition according to claim 11, wherein the glidant is selected from the group comprising colloidal silicon dioxide; silicates such as aluminium, calcium and magnesium; talc and the like and mixtures thereof.

18. The pharmaceutical composition according to claim 11, wherein the glidant is colloidal silicon dioxide.

19. A pharmaceutical composition, said composition comprising:
a) 0.1 to 60.0% of compound I or pharmaceutically acceptable salts thereof;
b) 0.1 to 40.0% of hydrochlorothiazide;
c) 1.0 to 90.0% of microcrystalline cellulose;
d) 0.1 to 30.0% of crospovidone;
e) 0.01 to 50.0% of sodium starch glycolate;
f) 0.01 to 20.0% of sodium stearyl fumarate; and
g) 0.01 to 15.0% of colloidal silicon dioxide.

20. The pharmaceutical composition according to claim 19, wherein the pharmaceutical composition is free of magnesium stearate.

21. The pharmaceutical composition according to claim 19, wherein the final dosage form is orally administrated in at least one of the forms consisting of tablets, bilayer tablets, capsules, powders and sachets.

22. The pharmaceutical composition according to claim 21, wherein the final dosage form is a tablet.

23. The pharmaceutical composition according to claim 22, wherein the tablet optionally comprise a coating layer.

24. The pharmaceutical composition according to claim 22, wherein the tablet is in the form of a bilayer tablet having the compound I or pharmaceutically acceptable salts thereof in one layer and hydrochlorothiazide in a second layer.

25. The pharmaceutical composition according to claim 24, wherein the bilayer tablet optionally comprise a coating layer.

26. The pharmaceutical composition according to claim 21, wherein the final dosage form is a capsule.

27. The pharmaceutical composition according to claim 19, wherein the final dosage form has a content uniformity of less than 2.0% RSD (Relative Standard Deviation).

28. The pharmaceutical composition according to claim 19, wherein the final dosage form has a content uniformity of preferably less than 1.0% RSD.

29. The pharmaceutical composition according to claim 1, wherein the hardness of the tablet is between 5 to 300 Newton.

30. The pharmaceutical composition according to claim 1, wherein the hardness of the tablet is between 20 to 150 Newton.

31. The pharmaceutical composition according to claim 1, wherein the friability of the tablet is less than 1%.

32. The pharmaceutical composition according to claim 1, wherein the pharmaceutical composition has a long term shelf-life of at least 24 months at ambient temperature, in its original packaging.

33. A wet granulation process for preparing a pharmaceutical composition according to claim 1, said process comprising the following steps:
a) dissolving Compound I and hydrochlorothiazide with half part of sodium starch glycolate and half part of crospovidone in organic solvent to form a solution;
b) while the solution is mixing microcrystalline cellulose is added and blended in a high-shear granulator to form granules;
c) sieving and drying the wet granules and milling the dried granules;
d) adding colloidal silicon dioxide and the rest of sodium starch glycolate and crospovidone and mixing them;
e) adding sodium stearyl fumarate to this mixture and blending them until obtaining a homogenous powder mixture; and
f) compressing the blended mixture to form tablets or filling the powder mixture into capsules.

34. A wet granulation process for preparing the pharmaceutical composition according to claim 1, said process comprising the following steps:
a) dissolving Compound I and hydrochlorothiazide with microcrystalline cellulose, half part of sodium starch glycolate and half part of crospovidone in organic solvent to form a solution in a fluid-bed granulator;
b) sieving and drying the wet granules and milling the dried granules;
c) adding colloidal silicon dioxide and the rest of sodium starch glycolate and crospovidone and mixing them;
d) adding sodium stearyl fumarate to this mixture and blending them until obtaining a homogenous powder mixture; and
e) compressing the blended mixture to form tablets or filling the powder mixture into capsules.

35. A dry granulation process for preparing the pharmaceutical composition according to claim 1, said process comprising the following steps:
a) mixing Compound I and hydrochlorothiazide with half part of microcrystalline cellulose, half part of sodium starch glycolate, half part of crospovidone and half part of colloidal silicon dioxide;
b) pressing them with the help of a compactor;
c) sieving these powder mixture;
d) adding the rest of microcrystalline cellulose, sodium starch glycolate, crospovidone and colloidal silicon dioxide and mixing them;
e) adding sodium stearyl fumarate to this mixture and blending them until obtaining a homogenous powder mixture; and
f) compressing the blended mixture to form tablets or filling the powder mixture into capsules.

36. A direct compression process for preparing the pharmaceutical composition according to claim 1, said process comprising the following steps:
a) mixing Compound I and hydrochlorothiazide with microcrystalline cellulose, sodium starch glycolate, crospovidone and colloidal silicon dioxide for 15 to 30 min;
b) adding sodium stearyl fumarate to this mixture and blending them until obtaining a homogenous powder mixture; and
c) compressing the final powder mixture to form tablets or filling the powder mixture into capsules.

37. The pharmaceutical composition according to claim 1, for preventing or treating hypertension in mammals, particularly in humans.
